(19) United States
(12) Patent Application Publication
(10) Pub. No.: US 2026/0265444 A1
(43) Pub. Date: Sep. 10, 2026

(54) FORMULATIONS BASED ON BIO-SOURCED POLYOLS

(71) Applicant: ARKEMA FRANCE, Puteaux (FR)

(72) Inventors: Clémentine CHAMPAGNE, Pierre-Benite, Cedex (FR); Anne PIGAMO, Pierre-Benite, Cedex (FR)

(73) Assignee: ARKEMA FRANCE, Puteaux (FR)

(21) Appl. No.: 19/152,411

(22) PCT Filed: Feb. 5, 2024
(86) PCT No.: PCT/FR2024/050140

§ 371 Date: Jul. 30, 2025

(30) Foreign Application Priority Data
Feb. 7, 2023 (FR) FR2301131

(51) Int. Cl. C08G 18/54; C08G 65/26; C09D 175/08; C09J 175/08; C09K 3/10

(52) U.S. Cl. CPC C08G 18/546; C08G 65/2612; C09D 175/08; C09J 175/08; C09K 3/1021; C09K 2200/065

(57) ABSTRACT

The present invention relates to formulations for obtaining polymer materials comprising an at least difunctional reactant capable of reacting with a polyol and a polyol capable of reacting with said at least difunctional reactant, said polyol being an at least partially biobased aromatic polyol. The invention also relates to the use of said formulations for the manufacture of polyurethanes, polyesters, polycarbonates or epoxy resins, intended for the production of coatings in general, paints, elastomers, sealants and adhesives.

### CROSS-REFERENCE TO RELATED APPLICATIONS

This application is the U.S. National Phase of PCT International Application No. PCT/FR2024/050140, filed Feb. 5, 2024, which claims priority to French Patent Application No. FR2301131, filed Feb. 7, 2023, the contents of these applications being incorporated by reference herein in their entireties for all purposes.

### FIELD OF THE INVENTION

The invention relates to formulations based on biobased polyols that can be used in the production of polymer resins. The present invention relates more specifically to formulations based on biobased polyols that can be used in the field of polymers in general and more particularly in the production of polyurethane, polyester, polycarbonate, epoxy, urethane-(meth)acrylate, polyester-(meth)acrylate, epoxy-(meth)acrylate and polyether-(meth)acrylate resins, and also silicone derivatives thereof and the like.

### BACKGROUND OF THE INVENTION

Formulations based on polyol(s) are currently very widely used and the polyols used are of very diverse types, depending on the uses for which they are intended, whether in the field of polymers, for example as polymerization reagents, wetting additives, plasticizers, dispersants, smoothing agents, coalescing agents, emulsifiers, reactive diluents and the like, in various fields, such as for example paints, coatings, resins, elastomers, sealants, adhesives, flexible and rigid foams, to name but the main ones thereof.

In order to further improve the properties of polyol formulations, the products in which they are used, and in particular in order to reduce their environmental impact, the industry is constantly seeking polyol-based formulations that have properties compatible with the intended uses, while favoring biobased raw materials.

There are already numerous formulations on the market which are based on biobased polyols, such as biobased polyethers, biobased polyesters from which they are derived, biobased aromatic polyols, and the like, to name but a few typical but nonlimiting examples.

Depending on the intended fields of application, there are however many difficulties associated with the use of such biobased polyol formulations, for example when they are used for the manufacture of polyurethane, as indicated for example in ACS, Sustainable Chem. Eng., (2021), 9, 10664-10677. Among these difficulties, those encountered most often and most generally are the following:
difficulties in supplying raw materials to manufacture polyols which compete with the food chain,
problems of reproducibility during the preparation of the polyols,
problems of reactivity of the biobased polyols which is often low,
low miscibility of these biobased polyol formulations with other ingredients necessary for the manufacture of materials, for example low miscibility with certain isocyanates and other comonomers and/or oligomers, other polyols, surfactants, catalysts or blowing agents, thus making them difficult to use or limiting their uses.

Formulations based on biobased polyols are nowadays prepared from natural oils, mainly vegetable or animal oils, among which mention may be made, as nonlimiting examples, of castor oil, palm oil and tall oil.

Other formulations based on biobased polyols are for example described in document CN102660014 A, in which an ethoxylated/propoxylated cardanol is involved in a Mannich reaction in order to prepare a polyurethane material. In document US2012129963, as in document WO2008017476, a non-alkoxylated cardanol-formaldehyde resin is used to synthesize a polyurethane material.

In document WO2018172222, polyols of phenolic type are used, without any notion of biobased carbon atoms being indicated. Ethoxylated and propoxylated phenol-formaldehyde resins are also described. And in document CN103073689, polyols are disclosed which have a very high hydroxyl value, which can be troublesome in certain specific applications.

There is therefore a need for formulations based on compatible and versatile biobased polyols to be incorporated into compositions for obtaining materials, and in particular for obtaining polymers.

### SUMMARY OF THE INVENTION

Thus, and according to a first aspect, the invention relates to a formulation for obtaining a polymer material comprising:
an at least difunctional reactant capable of reacting with a polyol,
a polyol capable of reacting with said at least difunctional reactant, said polyol:
   being an aromatic polyol
   having a hydroxyl value of less than or equal to 200 mg KOH g⁻¹,
   a number-average functionality of greater than or equal to 2, and
   a content of biomass-derived atoms of greater than 30%.

The formulation according to the present invention is very particularly suitable for obtaining polymer materials chosen from polyurethane, polyester, polycarbonate, epoxy, urethane-(meth)acrylate, polyester-(meth)acrylate, epoxy-(meth)acrylate and polyether-(meth)acrylate resins, and also silicone derivatives thereof and the like, to name but the main polymer materials which can be obtained from the formulation according to the present invention. The formulation of the present invention is very particularly suitable for the preparation of polyurethanes.

It should be understood that the formulation of the present invention, which comprises an at least partially biobased aromatic polyol, allows the preparation of polymers in which said aromatic polyol defined above and an at least difunctional reactant are involved.

Among the difunctional reactants which can be used in the formulation of the present invention, mention may be made, as nonlimiting examples, of isocyanates that are at least difunctional, carboxylic acids that are at least difunctional, epoxies that are at least difunctional, and the like. Mixtures of reactants that are at least difunctional may be envisaged in the formulation of the present invention.

### DETAILED DESCRIPTION OF THE INVENTION

According to a preferred embodiment, the formulation of the present invention is very particularly suitable for the manufacture of polyurethanes, and the at least difunctional reactant is chosen from isocyanates that are at least difunctional, preferably from difunctional isocyanates and multifunctional isocyanates.

The isocyanates which can be used in the context of the present invention are of any type, well known to those skilled in the art, and in particular are organic isocyanates and more particularly difunctional organic isocyanates. Nonlimiting examples of such diisocyanates include aliphatic diisocyanates having a hydrocarbon group comprising up to 18 carbon atoms, cycloaliphatic diisocyanates having a hydrocarbon group comprising up to 15 carbon atoms, aromatic diisocyanates having a hydrocarbon group comprising from 6 to 15 carbon atoms and arylaliphatic diisocyanates having a hydrocarbon group comprising from 8 to 15 carbon atoms. It is clearly understood that two or more different at least difunctional isocyanates may be included as a mixture in the formulation of the present invention, in any proportion.

Preferred diisocyanates are toluene-2,4-diisocyanate, toluene-2,6-diisocyanate, diphenylmethane diisocyanates, notably diphenylmethane-4,4′-diisocyanate, polymethylene polyphenyl isocyanates, and mixtures of two or more thereof in any proportion. Modified isocyanates that are at least difunctional, such as those comprising one or more carbodiimide groups, urethane groups, isocyanurate groups, urea groups and biuret groups, may also be suitable.

The polyol capable of reacting with said at least difunctional reactant in the formulation of the present invention is an aromatic polyol as indicated above. An “aromatic polyol” is understood to mean a polyol comprising at least one aromatic function, preferably an oligomer or polymer, the repeating unit of which comprises at least one, two or three aromatic function(s), preferably, one or two aromatic function(s), and more preferably one aromatic function.

In addition, the polyol capable of being used in the formulation of the invention has a hydroxyl value (OHV) of less than or equal to 200 mg KOH g⁻¹, preferably less than or equal to 160 mg KOH g⁻¹, more preferably less than or equal to 140 mg KOH g⁻¹, advantageously less than or equal to 120 mg KOH g⁻¹, very advantageously less than or equal to 100 mg KOH g⁻¹. In one embodiment of the invention, the hydroxyl value is equal to or greater than 30 mg KOH g⁻¹, preferably equal to or greater than 50 mg KOH g⁻¹. According to one quite preferred embodiment of the invention, the polyol capable of being used in the formulation of the invention has a hydroxyl value equal to or greater than 30 mg KOH g⁻¹ and less than or equal to 200 mg KOH g⁻¹, more preferably equal to or greater than 30 mg KOH g⁻¹ and less than or equal to 160 mg KOH g⁻¹, better still equal to or greater than 30 mg KOH g 1 and less than or equal to 140 mg KOH g⁻¹, typically equal to or greater than 30 mg KOH g⁻¹ and less than or equal to 120 mg KOH g⁻¹ or else equal to or greater than 30 mg KOH g⁻¹ and less than or equal to 100 mg KOH g⁻¹. According to yet another embodiment, the polyol capable of being used in the formulation of the invention has a hydroxyl value equal to or greater than 50 mg KOH g⁻¹ and less than or equal to 200 mg KOH g⁻¹, more preferably equal to or greater than 50 mg KOH g⁻¹ and less than or equal to 160 mg KOH g⁻¹, better still equal to or greater than 50 mg KOH g⁻¹ and less than or equal to 140 mg KOH g 1, typically equal to or greater than 50 mg KOH g⁻¹ and less than or equal to 120 mg KOH g⁻¹ or else equal to or greater than 50 mg KOH g⁻¹ and less than or equal to 100 mg KOH g⁻¹.

The hydroxyl value is a parameter well known to those skilled in the art and can be determined according to the standard DIN 53240-2.

The polyol capable of being used in the formulation of the invention also has a number-average functionality of greater than or equal to 2, preferably greater than or equal to 3. The lower the average functionality of the polyol, the more flexible the structures of the resulting polymers will be. Conversely, the higher the average functionality of the polyol, the more rigid the structures of the resulting polymers will be.

The “number-average functionality” is understood to mean the average number of hydroxyl functions (OH functions) per mole of polyol. More precisely, the number-average functionality (F_{OH}) is calculated according to the following formula:

$${F}_{OH} = ( {Mw}_{P} / {Mw}_{R} ) * {n}_{OH}$$

wherein:
Mw_{P} represents the number-average molar mass of the aromatic polyol,
Mw_{R} represents the molar mass of the repeating unit in the aromatic polyol, and
n_{OH} represents the number of hydroxyl functions present on the repeating unit,
   Mw_{P} in determined by size exclusion chromatography and Mw_{R} and n_{OH} being able to be determined by any analytical means well known to those skilled in the art, for example by NMR analysis.

In one embodiment, the number-average functionality is generally less than 1000, more often less than 500, preferably less than 100, more preferably less than 50 and advantageously less than 20. In a more preferred embodiment, the polyol has a number-average functionality of between 2 and 1000, preferably between 2 and 500, more preferably between 2 and 100, better still between 2 and 50, most often between 2 and 20, for example, between 3 and 20, limits included.

Finally, the polyol capable of being used in the formulation of the invention is at least partly biobased and more specifically said polyol comprises a content of biomass-derived atoms of greater than 30%, as indicated above and preferably of greater than 40%, the content measured according to the standard NF EN 16785-1 (January 2016).

In a preferred embodiment, the aromatic polyol of the formulation of the present invention is an oligomer or a polymer obtained by polycondensation of an aromatic phenol or aromatic phenol derivative with at least one compound chosen from aldehydes and ketones. An aromatic phenol is understood to mean aromatic phenols optionally substituted with one or more hydrocarbon chains. Preferred examples of such aromatic phenols are aromatic phenols which are at least partially or completely biobased, such as cardanol, cardol or methylcardol.

An “aromatic phenol derivative” is understood to mean the derivatives of the aromatic phenols described above, and in particular the alkoxylates of the aromatic phenols described above, that is to say aromatic phenols in which at least one hydroxy function is substituted with a (poly)ethoxy, (poly)propoxy, (poly)butoxy or (poly)tetramethyleneoxy chain, and the like, it being possible for the chain to comprise from 1 to 100, preferably from 1 to 50 repeating units, derived from aromatic phenols obtained according to techniques well known to those skilled in the art and for example by reaction of said aromatic phenols with one or more moles of a compound of epoxy or oxirane type, and preferably and respectively from among ethylene oxide, propylene oxide, butylene oxide and tetrahydrofuran.

The aldehydes and ketones which can be used to obtain an oligomer or polymer of aromatic phenol as indicated above may be of any type well known to those skilled in the art and in particular, and preferably those chosen from formaldehyde, and also any aldehyde containing a hydrocarbon chain comprising from 2 to 20 carbon atoms, preferably from 2 to 16 carbon atoms, dimethyl ketone and also any ketone containing a hydrocarbon chain comprising from 4 to 20 carbon atoms, preferably from 4 to 16 carbon atoms.

Aromatic phenol oligomers and polymers in which two aromatic rings are separated by a single carbon atom, which is itself unsubstituted or optionally substituted with one or two hydrocarbon chains comprising from 2 to 20 carbon atoms, preferably from 2 to 16 carbon atoms, are very particularly preferred.

Examples of aromatic polyols which have proved particularly suitable for the needs of the present invention are the oligomers and polymers obtained by polycondensation of an aromatic phenol or aromatic phenol derivative with formaldehyde. Among these aromatic polyols, preference is very particularly given to those chosen from cardanol-formaldehyde resins and alkoxylated cardanol-formaldehyde resins, where the term “alkoxylated” includes the terms ethoxylated, propoxylated and butoxylated, and also the term (poly)tetramethylene ether, obtained for example by reaction with tetrahydrofuran, as indicated above. Aromatic phenols which are very particularly preferred are alkoxylated cardanol-formaldehyde resins, and better still ethoxylated and/or propoxylated cardanol-formaldehyde resins.

Such alkoxylated cardanol-formaldehyde resins are well known to those skilled in the art and are commercially available or easily prepared using known procedures. For the purposes of the invention, alkoxylated cardanol-formaldehyde resins which are biobased and which comprise a content of biomass-derived atoms of greater than 30%, as indicated above, and preferably of greater than 40%, measured according to the standard NF EN 16785-1 (January 2016) are very particularly preferred.

The molar proportion of the reactive functions of the polyol(s) with respect to the reactive functions of the at least difunctional reactant(s) capable of reacting with said polyol(s) may vary to a large extent depending on the anticipated polymerization product and depending on the intended use for said anticipated polymerization product. However, a molar proportion of between 0.7 and 1 is preferred, and more preferably the molar proportion is between 0.8 and 1.

The formulation of the present invention may also comprise other components well known to those skilled in the art, among which mention may be made, in a completely nonlimiting manner, of other polyols, other at least difunctional reactants capable of reacting with the polyol(s), rheological agents, dyes, preservatives, catalysts, foaming agents, antifoaming agents, surfactants, flame retardants, antioxidants, compatibilizers, and the like, and mixtures of two or more thereof.

Examples of additional polyols which have proved particularly suitable for the needs of the present invention are, in a nonlimiting manner, polyether polyols, for example those obtained by condensation of an alkylene oxide or a mixture of alkylene oxides with glycerol, ethylene glycol, trimethylolpropane, pentaerythritol, neopentyl glycol or isosorbide, polyester polyols, for example those obtained from polycarboxylic acids, in particular oxalic acid, malonic acid, succinic acid, adipic acid, maleic acid, fumaric acid, isophthalic acid, terephthalic acid, with glycerol, ethylene glycol, trimethylolpropane, pentaerythritol, neopentyl glycol and the like.

Polyether polyols obtained by addition of alkylene oxides, in particular ethylene oxide and/or propylene oxide, to aromatic amines, in particular the mixture of 2,4-toluenediamine and 2,6-toluenediamine, may also be suitable as additional polyols which may be added to the formulation of the present invention.

The aromatic polyols described above have very many advantages and in particular that of exhibiting compatibility with any other polyols present in the formulation and more generally with all the other components of the formulation according to the invention, while being at least partially biobased.

According to a second aspect, the invention relates to the use of an aromatic polyol as has just been described above for the manufacture of polymers, in particular with one or more reactants that are at least difunctional, and optionally one or more additives chosen from wetting additives, plasticizers, dispersants, smoothing agents, coalescing agents, emulsifiers and reactive diluents, to mention only the main additives commonly used and well known to those skilled in the art.

The use according to the invention finds numerous applications in a wide variety of fields, among which mention may be made, in a nonlimiting manner, of the following industries: polymer production, production of coatings in general, paint production, production of elastomers, or else sealants or adhesives, to mention only the main production processes of interest. According to one embodiment, the polyols defined above are very particularly suitable as a component of a formulation according to the invention intended for the manufacture of polymers, and preferably for the manufacture of polyurethane, polyester, polycarbonate, epoxy, urethane-(meth)acrylate, polyester-(meth)acrylate, epoxy-(meth)acrylate, polyether-(meth)acrylate resins, and also silicone derivatives thereof, and very advantageously for the preparation of polyurethanes.

The polyurethane obtained from the formulation of the present invention finds numerous possible uses in various fields of industry, among which may be made, in a nonlimiting manner, of the fields of coatings in general, paints, adhesives, sealants and elastomers. The polyurethanes thus produced may be in any form well known to those skilled in the art and in particular in liquid form, in the form of flexible foam or rigid foam, and the like.

The invention is now illustrated with the aid of the examples which follow and which in no way limit the invention, the scope of which is defined by the claims appended to the present description.

### Examples

### Example 1: Synthesis of an Aromatic Polyol

### Step 1:

A 4-liter three-necked round-bottom flask inerted with nitrogen is charged with cardanol (1854 g, 6.2 mol) and para-formaldehyde (146 g, 4.9 mol) at room temperature into. The medium is heated to 80° C. and then 8.9 g of 32% HCl are added. The reaction is maintained for 30 minutes at 80° C. and is then heated to 110° C. (distillation assembly). The reaction is maintained for 4 hours and then a gradual vacuum is produced (15 kPa) and the reaction is maintained for 1 h 30 min at 120° C. After returning to atmospheric pressure and to 80° C., 11.1 g of KOH are added. After maintaining stirring for one hour, the medium is cooled and the product is recovered without further treatment. The polymer obtained has a number-average weight of 1300 g mol-1, determined by NMR.

### Step 2:

1200 g of the product obtained in step 1 and 10.6 g of KOH are introduced into an alkoxylation reactor. They are dehydrated under vacuum at 110° C. for one hour to achieve a water quantity of less than 0.1%. The reactor is purged under nitrogen and the stirring is set at 1100 rpm. The reaction medium is heated to 155-160° C. Ethylene oxide (1293 g) is introduced slowly while cooling in order to keep the reactor at the set temperature. Once all the ethylene oxide has been introduced and the pressure stabilized, the medium is cooled to 70° C. and 6.8 g of acetic acid are added.

The polymer obtained has a hydroxyl value of 84 mg KOH g⁻¹, a number-average functionality of 4.2 and a content of biobased atoms of 45%. This polyol is called Polyol A, and it is in accordance with the polyol of the formulation of the present invention.

### Example 2: Miscibility in a Polyol Formulation

Formulated polyol compositions containing a polyol mixture, a blowing agent and a catalyst mixture are prepared. All the formulations have the same amounts of blowing agent and catalyst. The polyols used have the characteristics shown in table 1 below:

TABLE 1

| | | | Content of |
|---|---|---|---|
| | | | biobased atoms |
| Polyol | OHV (mg KOH g⁻¹) | Functionality | (%) |
| Polyol M420⁽¹⁾ | 415 | 4 | 0 |
| Polyol A⁽²⁾ | 84 | 4.2 | 45 |
| Polyol B⁽³⁾ | 440 | 4 | 78 |
| Polyol C⁽⁴⁾ | 475 | 4 | 60 |

⁽¹⁾Non-biobased polyol, sold by PCI (outside the invention)
⁽²⁾Biobased polyol A according to the invention, prepared according to example 1
⁽³⁾Biobased polyol B (outside the invention), GX9102 sold by Cardolite
⁽⁴⁾Biobased polyol C (outside the invention), GX9103 sold by Cardolite

Formulation 1 does not contain any biobased polyol. Formulation 2 replaces one of the polyols of formulation 1 with a biobased polyol A according to the invention. Formulations 3 and 4 also contain biobased polyols B and C in an amount equivalent to formulation 2, but the properties of which are not in accordance with the invention.

The formulated polyols comprising the additional components and the blowing agent are presented in table 2 below and are expressed in pphp.

TABLE 2

| Formulation # | 1 | 2 | 3 | 4 |
|---|---|---|---|---|
| Terate HT 5510⁽⁵⁾ | 55 | 55 | 55 | 55 |
| Voranol CP 450⁽⁶⁾ | 22.5 | 22.5 | 22.5 | 22.5 |
| Polyol M420 | 22.5 | | | |
| Polyol A | | 22.5 | | |
| Polyol B | | | 22.5 | |
| Polyol C | | | | 22.5 |
| (1,1,3,3-tetramethylguanidine)⁽⁷⁾ | 7.23 | 6.42 | 7.31 | 7.41 |
| DABCO T120⁽⁸⁾ | 0.29 | 0.26 | 0.29 | 0.29 |
| Tegostab B84711⁽⁹⁾ | 1.47 | 1.5 | 1.48 | 1.5 |
| Tris(1-chloro-2-propyl) | 19.4 | 17.2 | 19.62 | 19.91 |
| phosphate⁽¹⁰⁾ | | | | |
| Added water | 1.66 | 1.47 | 1.68 | 1.7 |
| 1233zd | 14.45 | 12.82 | 14.6 | 14.82 |
| TOTAL B-SIDE | 144.5 | 139.67 | 144.98 | 145.63 |

⁽⁵⁾Polyester polyol sold by Invista (OHV = 250-265)
⁽⁶⁾Polyether polyol sold by Dow
⁽⁷⁾Amine catalyst sold by Sigma-Aldrich
⁽⁸⁾Metal catalyst sold by Evonik
⁽⁹⁾Foam stabiliser sold by Dow
⁽¹⁰⁾Flame retardant (tris(1-chloro-2-propyl) phosphate)

The formulations are prepared by successive addition of the desired amount of each component and left to stand for 72 hours at room temperature. The possible appearance of several phases is observed, as shown in table 3 below.

TABLE 3

| Formulation # | 1 | 2 | 3 | 4 |
|---|---|---|---|---|
| Miscibility | miscible | miscible | two-phase | two-phase |

The formulations are prepared and aged for 7 days at 50° C. Polyurethane foams are prepared using the formulations described in table 2 above and by adding the isocyanates as indicated in table 4 below.

TABLE 4

| Formulation # | 1 | 2 | 3 | 4 |
|---|---|---|---|---|
| Ongronat 2100⁽¹¹⁾ | 143.52 | 115.94 | 146.17 | 149.85 |
| ROH index⁽¹²⁾ | 119 | 119 | 119 | 119 |
| B/A⁽¹³⁾ | 1.01 | 1.20 | 0.99 | 0.97 |
| Content of blowing agent | 5 | 5 | 5 | 5 |
| (wt %) | | | | |

⁽¹¹⁾Isocyanate sold by BorsodChem
⁽¹²⁾molar ratio of [(NCO functions)/(OH functions)] × 100
⁽¹³⁾weight ratio (formulated polyols/isocyanates)

The foam growth kinetics are presented in table 5 below. The free growth reaction profile is defined by the terms and definitions explained in the standard NF EN 14315-1.

Cream time is the time elapsed between the start of the procedure for stirring the mixed components and the moment when the foam begins to grow (measured in seconds).

String time is the time elapsed between the start of the procedure for stirring the mixed components and the moment when, by means of a rod applied to the surface of the foam, a polymer chain can be extracted from the surface of the foam (measured in seconds).

Tack-free time is the time elapsed between the start of the procedure for stirring the mixed components and the moment when, by means of a rod applied to the surface of the foam, it is established that the surface is no longer tacky (measured in seconds).

TABLE 5

| Formulation # | 1 | 2 | 3 | 4 |
|---|---|---|---|---|
| Cream time (s) | 5 | 4 | 5 | 6 |
| String time (s) | 12 | 12 | 26 | 25 |
| Tack-free time (s) | 16 | 15 | 37 | 34 |

It is noted that formulation 2 has an identical reactivity to formulation 1. Formulations 3 and 4 have significantly lower reactivities and cannot be used as a replacement for formulation 1.

1. A formulation for obtaining a polymer material comprising an at least difunctional reactant capable of reacting with a polyol and a polyol capable of reacting with the at least difunctional reactant, the polyol:
being an aromatic polyol
having a hydroxyl value of less than or equal to 200 mg KOH g⁻¹,
a number-average functionality of greater than or equal to 2, and
a content of biomass-derived atoms of greater than 30%.

2. The formulation as claimed in claim 1, for obtaining polymer materials chosen from polyurethane, polyester, polycarbonate, epoxy, urethane-(meth)acrylate, polyester-(meth)acrylate, epoxy-(meth)acrylate and polyether-(meth)acrylate resins and also silicone derivatives thereof.

3. The formulation as claimed in claim 1, wherein the polyol capable of reacting with the at least difunctional reactant is a polyol comprising at least one aromatic function, the repeating unit of which comprises at least one, two or three aromatic function(s).

4. The formulation as claimed in claim 1, wherein the polyol capable of being used in the formulation of the invention has a hydroxyl value (OHV) of less than or equal to 200 mg KOH g⁻¹.

5. The formulation as claimed in claim 1, wherein the polyol has a number-average functionality of greater than or equal to 2.

6. The formulation as claimed in claim 1, wherein the polyol has a number-average functionality of between 2 and 1000, limits included.

7. The formulation as claimed in claim 1, wherein the polyol comprises a content of biomass-derived atoms of greater than 40%.

8. The formulation as claimed in claim 1, wherein the aromatic polyol is chosen from alkoxylated cardanol-formaldehyde resins.

9. The formulation as claimed in claim 1, wherein the difunctional reactant is chosen from isocyanates that are at least difunctional, carboxylic acids that are at least difunctional, and epoxies that are at least difunctional.

10. The formulation as claimed in claim 1, for manufacturing polyurethanes, in which the at least difunctional reactant is chosen from isocyanates that are at least difunctional.

11. The formulation as claimed in claim 1, wherein the isocyanates are difunctional organic isocyanates chosen from aliphatic diisocyanates having a hydrocarbon group comprising up to 18 carbon atoms, cycloaliphatic diisocyanates having a hydrocarbon group comprising up to 15 carbon atoms, aromatic diisocyanates having a hydrocarbon group comprising from 6 to 15 carbon atoms and arylaliphatic diisocyanates having a hydrocarbon group comprising from 8 to 15 carbon atoms, and mixtures of two or more thereof in any proportion.

12. A method for manufacturing polyurethane, polyester, polycarbonate, epoxy, urethane-(meth)acrylate, polyester-(meth)acrylate, epoxy-(meth)acrylate and polyether-(meth)acrylate resins and also silicone derivatives thereof, comprising adding the formulation as claimed in claim 1 as a component during preparation of the resins and silicone derivatives thereof.

13. The method as claimed in claim 12, for the production of coatings in general, paints, elastomers, sealants and adhesives.
